(19) United States
(12) Patent Application Publication
(10) Pub. No.: US 2021/0161879 A1
(43) Pub. Date: Jun. 3, 2021

(54) FLECAINIDE COMBINATION AND CONTROLLED-RELEASE FORMULATIONS FOR TREATING HEART DISEASES

(71) Applicant: ALSAR LTD PARTNERSHIP, Plantation, FL (US)

(72) Inventor: Robert FISHEL, Delray Beach, FL (US)

(73) Assignee: ALSAR LTD PARTNERSHIP, Plantation, FL (US)

(21) Appl. No.: 17/168,098

(22) Filed: Feb. 4, 2021

Related U.S. Application Data
(63) Continuation of application No. PCT/US2020/033219, filed on May 15, 2020.
(60) Provisional application No. 62/860,919, filed on Jun. 13, 2019.
(60) Provisional application No. 62/849,557, filed on May 17, 2019.
(60) Provisional application No. 62/849,415, filed on May 17, 2019.
(60) Provisional application No. 62/849,366, filed on May 17, 2019.

(51) Int. Cl. A61K 31/4458; A61K 31/138; A61K 31/165; A61K 31/137; A61K 31/554; A61K 31/7048; A61K 9/48

(52) U.S. Cl. CPC A61K 31/4458; A61K 31/138; A61K 31/165; A61K 9/48; A61K 31/554; A61K 31/7048; A61K 31/137

(57) ABSTRACT

The invention relates to flecainide formulations and to methods of their administration. Specifically, the invention relates to combination formulations of a flecainide and a rate control agent for treating various heart diseases, and to controlled-release flecainide formulations, including such formulations in combination with rate control agents.

### CROSS-REFERENCE TO RELATED APPLICATIONS

This application claims benefit of U.S. Provisional patent Application No. 62/849,366, filed May 17, 2019, U.S. Provisional patent Application No. 62/860,919, filed Jun. 13, 2019, U.S. Provisional patent Application No. 62/849,415, filed May 17, 2019, and U.S. Provisional Patent Application No. 62/849,557, filed May 17, 2019, each of which is incorporated by reference herein in its entirety.

### FIELD OF THE INVENTION

The invention relates to flecainide formulations and to methods of their administration. Specifically, the invention relates to combination formulations of a flecainide and a rate control agent for treating various heart diseases, and to controlled-release flecainide formulations, including such formulations in combination with rate control agents.

### BACKGROUND OF THE INVENTION

Supraventricular tachycardia (SVT), atrial fibrillation, and atrial flutter are serious heart diseases often treated by Flecainide. Flecainide and other type IC anti-arrhythmic drugs typically cannot be given solely for many type arrhythmias, however, as they have the potential to paradoxically increase AV nodal conduction while also slowing but not terminating the underlying atrial arrhythmia

An example will better illustrate this phenomena. If a patient develops right atrial flutter (i.e., tricuspid annular dependent right atrial reentry) the rate in the atria is typically close to 300 BPM. The atrial impulse needs to transit across the AV node in order to cause ventricular contraction. AV nodal physiology prevents conduction of an atrial rate as fast as 300 BPM. This is normal AV nodal physiology and can be considered a type of natural circuit breaker. Ventricular rates of 300 BPM are too fast to allow mechanical contraction of the heart and typically cause cardiac arrest and death. The AV node typically will only conduct every other or every third impulse to the ventricles so during a typical episode of atrial flutter whereby the atrial rate is 300 BPM, the ventricular rate is a fixed fraction of that rate, usually 150 BPM or 100 BPM.

Flecainide and other similar anti-arrhythmic drugs can both slow the rate of tachycardia in the atria and simultaneously increase AV nodal conduction. The net effect of these actions is that dosing of flecainide alone can cause the atrial rate to slow to 200 BPM and, by accelerating AV nodal conduction, can allow for 1:1 atria to ventricular conduction thus producing a ventricular rate of 200 BPM worsening the clinical status of the patient. Thus, these medications can be supplemented with rate control agents, such as beta blockers, calcium channel blockers, or digitalis, which act to slow AV nodal conduction, preventing this paradoxical increase in the ventricular rate and potential worsening of the patient's condition.

Typically, rate control agents would be given prior to administration of flecainide and similar anti-arrhythmic medications so that the patient is protected from rapid tachycardia secondary to the above phenomena.

Complicating treatment success, however, medications such as flecainide often need to be dosed two or more times daily and therefore need to be supplemented separately with rate control agents for each such dosing, thereby causing both inconvenience and non-compliance.

Accordingly, there exists a need for improved flecainide formulations and methods that overcome the various shortcomings seen in the art with currently known therapies.

### SUMMARY OF THE INVENTION

In an aspect, the invention provides a combination therapeutic method to treat a heart disease in a subject. In an embodiment, the method includes administering to the subject a therapeutically effective amount of a flecainide in combination with a rate control agent. In embodiments, the use of a rate control agent in combination with flecainide enhances the release rate of flecainide to treat a heart disease.

In some embodiments, a flecainide is co-administered with a rate control agent. In other embodiments, a flecainide is administered independently from the administration of a rate control agent.

In an aspect, the invention also provides a pharmaceutical composition that comprises a therapeutically effective amount of a flecainide and a therapeutically effective amount of a rate control agent. In some embodiments, the rate control agent is present in said composition in an amount effective to control the release rate of flecainide to treat a heart disease.

In an aspect, the invention also provides a controlled-release pharmaceutical formulation comprising a flecainide. In an embodiment, the controlled-release formulation is a sustained-release formulation.

In an aspect, the invention also provides a method of manufacturing the controlled-release formulation of the invention, the method comprising intermixing flecainide with an effective amount of an excipient to form a mixture and configuring the mixture into a unit dosage form.

In yet another aspect, the invention provides a method of treatment comprising administering a controlled-release flecainide formulation of the invention to a subject in need thereof.

Other features and advantages of the present invention will become apparent from the following detailed description and examples. It should be understood, however, that the detailed description and the specific examples, while indicating embodiments of the invention are given by way of illustration only, as various changes and modifications within the spirit and scope of the invention will become apparent to those skilled in the art from this detailed description.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount. Thus, for example, reference to a formulation that comprises “about 70% flecainide by weight” will be understood as a reference to an amount of flecainide in the pharmaceutical formulation that is 70%±14% (i.e., between 56% and 84%) by weight, or preferably 70%±7% (i.e., between 63% and 77% by weight), or more preferably 70%±4% (i.e., between 66% and 74% by weight).

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The term “stereoisomers” refers to compounds that have identical chemical constitution, but differ as regards the arrangement of the atoms or groups in space. The term “enantiomers” refers to stereoisomers that are mirror images of each other that are non-superimposable.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

Conditions and disorders in a subject for which a particular drug or compound (such as a flecainide or a rate control agent) is said herein to be “indicated” are not restricted to conditions and disorders for which that drug or compound has been expressly approved by a regulatory authority, but also include other conditions and disorders known or reasonably believed by a physician to be amenable to treatment with that drug, compound, composition, formulation, or combination thereof.

Various embodiments provide pharmaceutical formulations that provide controlled-release of a flecainide. Such formulations can be configured in various ways and in a variety of dosage forms, such as tablets and capsules, to modify the release of the flecainide. For example, one type of controlled-release pharmaceutical formulation is a sustained-release flecainide pharmaceutical formulation. Sustained-release flecainide pharmaceutical formulations can contain a variety of excipients, such as controlled-release excipients (also referred to as release modifiers) and/or fillers that are selected and incorporated into the formulation in such a way as to slow the dissolution rate of the formulation (and thereby slow the dissolution and/or release of the flecainide) under in vivo conditions as compared to an otherwise comparable immediate-release formulation.

The term “immediate-release” is used herein to specify a formulation that is not configured to alter the dissolution profile of the active ingredient (e.g., flecainide). For example, an immediate-release pharmaceutical formulation may be a pharmaceutical formulation that does not contain ingredients that have been included for the purpose of altering the dissolution profile. An immediate-release formulation thus includes drug formulations that take less than 30 minutes for substantially complete dissolution of the drug in a standard dissolution test. A “standard dissolution test,” as that term is used herein, is a test conducted according to United States Pharmacopeia 24th edition (2000) (USP 24), pp. 1941-1943, using Apparatus 2 described therein at a spindle rotation speed of 100 rpm and a dissolution medium of water, at 37° C., or other test conditions substantially equivalent thereto.

The term “controlled-release” is used herein in its ordinary sense and thus includes pharmaceutical formulations that are combined with ingredients to alter their dissolution profile. A “sustained-release” formulation is a type of controlled-release formulation, wherein ingredients have been added to a pharmaceutical formulation such that the dissolution profile of the active ingredient is extended over a longer period of time than that of an otherwise comparable immediate-release formulation. A controlled-release formulation thus includes drug formulations that take 30 minutes or longer for substantially complete dissolution of the drug in a standard dissolution test, conditions which are representative of the in vivo release profile.

The term “orally deliverable” is used herein in its ordinary sense and thus includes drug formulations suitable for oral, including peroral and intra-oral (e.g., sublingual or buccal) administration. Preferred compositions are adapted primarily for peroral administration, e.g., for swallowing. Examples of preferred orally deliverable compositions include discrete solid articles such as tablets and capsules, which are typically swallowed whole or broken, with the aid of water or other drinkable fluid.

The term in vivo “absorption” is used herein in its ordinary sense and thus includes reference to the percentage of a flecainide or other drug (e.g. rate control agent) that enters the bloodstream, as conventionally calculated from data of a standard pharmacokinetic (PK) study involving oral administration of a single dose of a flecainide or other drug. It will be understood that PK data are subject to the usual variation seen in biological data, in accordance with standard statistical practice.

In one aspect, the composition of the invention comprises flecainide, which is well known in the art. Flecainide is in a group of drugs called Class IC anti-arrhythmics Flecainide (brand name Tambocor) is used to treat irregular heartbeats (arrhythmias), maintain a normal heart rate or slow an overactive heart. It relaxes the heart and improves its pumping action. The Food and Drug Administration (FDA) approved flecainide in 1985. It's sold as Tambocor by 3M Pharmaceuticals. Formulations of the invention can exhibit any of the release profiles and/or characteristics described herein.

Flecainide is well known and fully described in U.S. Pat. Nos. 9,750,734, 7,196,197, 6,599,922, 6,593,486, 6,538,138, and 6,316,627, and U.S. Patent Application Publications 20190008844, 20180028519, 20160158213, 20100184990, 20050059825, 20040220409, 20030032835, and 20020133013, all of which are incorporated by reference herein in their entireties.

### Beta Blockers

As described herein, embodiments of the invention include compositions that comprise rate control agents. In an aspect, the rate control agent can comprise a beta blocker. Beta blockers (also referred to as β-blockers or beta blocker drugs) are a class of medications that are predominantly used to manage abnormal heart rhythms, and to protect the heart from a second heart attack (myocardial infarction) after a first heart attack (secondary prevention). They are also widely used to treat high blood pressure (hypertension).

Beta blockers are competitive antagonists that block the receptor sites for the endogenous catecholamines epinephrine (adrenaline) and norepinephrine (noradrenaline) on adrenergic beta receptors, of the sympathetic nervous system.

Some block activation of all types of β-adrenergic receptors and others are selective for one of the three known types of beta receptors, designated β₁, β₂ and β₃ receptors. β₁-adrenergic receptors are located mainly in the heart and in the kidneys. β₂-adrenergic receptors are located mainly in the lungs, gastrointestinal tract, liver, uterus, vascular smooth muscle, and skeletal muscle. β₃-adrenergic receptors are located in fat cells.

In one embodiment, the beta blocker drug of the invention is a non-specific or non-selective beta blocker drug.

In another embodiment, the beta blocker drug of the invention is a specific or selective beta blocker drug. In one embodiment, the beta blocker drug of the invention specifically or selectively blocks the activation of β₁ receptor.

In another embodiment, the beta blocker drug of the invention specifically or selectively blocks the activation of β₂ receptor. In yet another embodiment, the beta blocker drug of the invention specifically or selectively blocks the activation of β₃ receptor.

Examples of a non-specific or non-selective beta blocker drug include, without limitation, propranolol, bucindolol, carteolol, carvedilol, labetalol, nadolol, oxprenolol, penbutolol, pindolol, sotalol, and timolol.

Examples of β₁-selective or β₁-specific beta blockers include, without limitation, acebutolol, atenolol, betaxolol, bisoprolol, celiprolol, metoprolol, nebivolol, and esmolol.

β₁-selective or β₁-specific beta blockers are also known as cardioselective beta blockers. In a preferred embodiment, the beta blocker drug is a β₁-selective or β₁-specific beta blocker.

Examples of β₂-selective or β₂-specific beta blockers include, without limitation, butaxamine and ICI-118,551.

Examples of β₃-selective or β₃-specific beta blockers include, without limitation, SR 59230A.

In one embodiment, the beta blocker drug is a β₁ selective antagonist and β₃ agonist agent. Example of such β₁ selective antagonist and β₃ agonist agent includes, without limitation, nebivolol.

Other examples of a beta blocker drug include, without limitation, bisoprolol, metoprolol, nadolol, betaxolol, bisoprolol, esmolol, alprenolol, bucindolol, levobunolol, medroxalol, mepindolol, metipranolol, propafenone (propafenone is a sodium channel blocking drug that also is a beta-adrenergic receptor antagonist), propranolol, sotalol, and timolol.

### Calcium Channel Blockers

As described herein, embodiments of the invention include compositions wherein a rate control agent comprises a calcium channel blocker.

Calcium channel blockers are well known in the art and fully described in U.S. Pat. Nos. 10,117,848; 9,132,200; 8,748,648; 8,318,721; 5,209,933; and 4,552,881, and U.S. Patent Application Publications 20150335628; 20140323529; and 20110098273, which are incorporated by reference herein in their entirety.

Calcium channel blockers (CCB) are medications that disrupt the movement of calcium (Ca²⁺) through calcium channels. Calcium channel blockers are particularly effective against large vessel stiffness, one of the common causes of elevated systolic blood pressure in elderly patients. Calcium channel blockers are also frequently used to alter heart rate, to prevent cerebral vasospasm, and to reduce chest pain caused by angina pectoris.

N-type, L-type, and T-type voltage-dependent calcium channels are present in the zona glomerulosa of the human adrenal gland, and calcium channel blockers can directly influence the biosynthesis of aldosterone in adrenocortical cells, with consequent impact on the clinical treatment of hypertension with these agents.

In one embodiment, calcium channel blockers are dihydropyridine (DHP) calcium channel blockers. Examples of dihydropyridine (DHP) calcium channel blockers include, without limitation, amlodipine (Norvasc), aranidipine (Sapresta), azelnidipine (Calblock), barnidipine (HypoCa), benidipine (Coniel), cilnidipine (Atelec, Cinalong, Siscard), clevidipine (Cleviprex), efonidipine (Landel), felodipine (Plendil), isradipine (DynaCirc, Prescal), lacidipine (Motens, Lacipil), lercanidipine (Zanidip), manidipine (Calslot, Madipine), Nicardipine (Cardene, Carden SR), nifedipine (Procardia, Adalat), nilvadipine (Nivadil), nimodipine (Nimotop), nisoldipine (Baymycard, Sular, Syscor), nitrendipine (Cardif, Nitrepin, Baylotensin), and pranidipine (Acalas).

In another embodiment, calcium channel blockers are non-dihydropyridine calcium channel blockers. Examples of non-dihydropyridine calcium channel blockers include, without limitation, phenylalkylamine and benzothiazepine. Examples of phenylalkylamine include, without limitation, verapamil (Calan, Isoptin), fendiline, and gallopamil Examples of benzothiazepine include, without limitation, diltiazem (Cardizem).

In some embodiments, calcium channel blockers are nonselective, which include, for example, without limitation, mibefradil, bepridil, flunarizine, fluspirilene, and fendiline.

Other examples of calcium channel blockers include, without limitation, Ziconotide peptide and Gabapentinoids, such as gabapentin and pregabalin.

In particular embodiments, calcium channel blockers are, for example, dihydropyridines (e.g. amlodipine), benzothiapines (e.g. diltiazem), and phenylalkylamines (e.g. verapamil), felodipine, nifedipine.

### Digitalis

As described herein, embodiments of the invention include compositions wherein a rate control agent comprises a digitalis. Digitalis is well known and fully described in, for example, U.S. Pat. Nos. 6,465,463; 5,545,623; 5,153,178; 4,436,828; 4,282,151; 4,133,949; and 3,997,525 and U.S. Patent Application Publications 20060205679; 20160206641; 20090209504; 20050026849; 20040082521; and 20040023967, all of which are incorporated by reference herein in their entireties.

In a particular embodiment, the digitalis is a digitalis glycoside. It is known in the art that digitalis glycosides are reversible allosteric inhibitors of Na⁺/K⁺-ATPase. Cardiac glycosides act through inhibition of Na⁺/K⁺ ATPase which subsequently causes the intracellular Ca²⁺ concentration ([Ca²⁺]) to increase. In medical practice, digitalis glycosides are administered at doses that produce a moderate degree of enzyme inhibition, for example, approximately 30%, in cardiac muscle. When the muscle cell membrane is depolarized by the action of cardiac glycosides, there are fewer uninhibited Na⁺/K⁺ ATPase enzymes available for the restoration of the Na⁺/K⁺ balance after muscle contraction. The remaining Na⁺/K⁺ ATPase enzymes which are not inhibited by cardiac glycosides will increase their rate of ion transport due to the high [Na⁺]i. For the muscle cell to respond correctly the next triggering nerve impulse, the Na⁺/K⁺ ionic gradient must be restored, although restoration of the gradient will take longer than it would if every Na⁺/K⁺ ATPase were available. This lag causes a temporary increase of [Na⁺]i. This temporary increase of [Na⁺]i causes Ca²⁺ to move into the cell through a Nat/Ca²⁺ ion channel. The Na⁺/Ca²⁺ ion channel allows Na to exit from the cell in exchange for Ca²⁺, or Ca²⁺ exit from the cell in exchange for Nat, depending on the prevailing Na and Ca²⁺ electrochemical gradients. In this way inhibition of the Na⁺/K⁺-ATPase by cardiac glycosides causes the Na⁺/Ca²⁺ exchange to partly reverse resulting in increased intracellular Ca²⁺, which in turn causes increased muscle contractility.

Examples of a digitalis glycoside include, for example, but not limited to oleandrin, neriifolin, odoroside A and H, ouabain (G-strophantin), cymarin, sarmentocymarin, periplocymarin, K-strophantin, thevetin A, cerberin, peruvoside, thevetosin, thevetin B, tanghinin, deacetyltanghinin, echujin, hongheloside G, honghelin, periplocin, strophantidol, nigrescin, uzarin, calotropin, cheiroside A, cheirotoxin, euonoside, euobioside, euomonoside, lancetoxin A and B, kalanchoside, bryotoxin A-C, bryophyllin B, cotiledoside, tyledoside A-D, F and G, orbicuside A-C, alloglaucotoxin, corotoxin, coroglaucin, glaucorin, scillarene A and B, scilliroside, scilliacinoside, scilliglaucoside, scilliglaucosidin, scillirosidin, scillirubrosidin, scillirubroside, proscillaridin A, rubelin, convalloside, convallatoxin, bovoside A, glucobovoside A, bovoruboside, antiarin A, helleborin, hellebrin, adonidin, adonin, adonitoxin, thesiuside, digitoxin, gitoxin, gitalin, digoxin, F-gitonin, digitonin, lanatoside A-C, bufotalin, bufotalinin, bufotalidin, pseudobufotalin, acetyl-digitoxin, acetyl-oleandrin, beta-methyldigoxin, and alpha-methyldigoxin.

In a particular embodiment, the digitalis glycoside is digitoxin or digoxin.

### Controlled and Sustained Release Formulations

In some embodiments described herein, sustained-release flecainide pharmaceutical formulations comprise one or more controlled-release excipients. In this context, the term “controlled-release” excipient is used herein in its ordinary sense and thus includes, as noted above, an excipient that is configured (e.g., incorporated into the formulation) in such a way as to control a dissolution profile of the drug, e.g., slow the dissolution of the flecainide in a standard dissolution test, as compared to an otherwise comparable pharmaceutical formulation that does not contain the controlled-release excipient. Examples of pharmaceutically acceptable controlled-release excipients include, without limitation, hydroxypropyl methylcellulose (HPMC), hydroxyethylcellulose, hydroxypropylcellulose (HPC), methylcellulose, ethylcellulose, cellulose acetate butyrate, cellulose acetate phthalate, hydroxypropylmethyl cellulose phthalate, microcrystalline cellulose, corn starch, polyethylene oxide, polyvinyl alcohol (PVA), polyvinylpyrrolidone (PVP), cross-linked PVP, polyvinyl acetate phthalate, polyethylene glycol, zein, poly-DL-lactide-co-glycolide, dicalcium phosphate, calcium sulfate, and mixtures thereof. In some embodiments, the controlled-release excipient comprises a sustained-release polymer, e.g., at least one of hydroxypropyl methylcellulose (HPMC), hydroxyethylcellulose, hydroxypropylcellulose (HPC), methylcellulose, ethylcellulose, cellulose acetate butyrate, cellulose acetate phthalate, hydroxypropylmethyl cellulose phthalate, microcrystalline cellulose, corn starch, polyethylene oxide, polyvinyl alcohol (PVA), polyvinylpyrrolidone (PVP), cross-linked PVP, polyvinyl acetate phthalate, polyethylene glycol, zein, poly-DL-lactide-co-glycolide (PLGA), and mixtures thereof. Controlled-release excipients may be referred to herein as release modifiers.

In certain embodiments, the controlled-release pharmaceutical formulation comprises flecainide dispersed in a wax matrix.

In some embodiments, the wax matrix comprises a controlled-release excipient, which is insoluble and erodible in water, including but not limited to, carnauba wax, stearyl alcohol, stearic acid, polyethylene glycol hydrogenated castor oil, castor wax, polyethylene glycol monostearate, and triglycerides.

In various embodiments, the controlled-release pharmaceutical formulation comprises flecainide dispersed in polymer matrix.

In some embodiments, the polymer matrix comprises a controlled-release excipient, which is water insoluble and inert in water, including but not limited to, ethyl cellulose, polyethylene, methyl acrylate-methacrylate copolymer, and polyvinyl chloride.

In other embodiments, the polymer matrix comprises a controlled-release excipient, which is hydrophilic and soluble in water, including but not limited to, cellulose derivatives (including, but not limited to, methylcellulose, hydroxyethyl cellulose, hydroxypropylmethyl cellulose (HPMC), sodium carboxymethyl cellulose (“sodium CMC”); non-cellulose polysaccharides (including, but not limited to sodium alginate, potassium alginate, agar, carrageen, xanthan gum, arabic gum, and caraia gum; galactomannose, guar gum, alfarroba gum); and acrylic acid polymers (including, but not limited to carboxypolymethylene).

In other embodiments, the controlled-release pharmaceutical formulation comprises flecainide dispersed in an encapsulated form.

In a matrix system, the drug is dispersed as solid particle within a porous matrix formed of a water insoluble polymer, such as polyvinyl chloride.

In various embodiments, the matrix system may be a slowly eroding matrix, including but not limited to waxes, glycerides, stearic acid, cellulosic materials. In some embodiments, a portion of the drug intended to have sustained action is combined with lipid or cellulosic material and then granulated.

In certain embodiments, the drug may be embedded in an inert plastic matrix. In embodiments, the drug may be granulated with an inert, insoluble matrix, including but not limited to polyethylene, polyvinyl acetate, polystyrene, polyamide or polymethacrylate.

In certain embodiments, the drug may be coated on its surface with a material, such as with a polymer) that retards penetration by the dispersion fluid. The coating may be performed by microencapsulation, a process in which a relatively thin coating is applied to small particles of solid or droplets of liquids and dispersion. In embodiments, polymers, include but are not limited to, polyvinyl alcohol, polyacrylic acid, ethylcellulose, polyethylene, polymethacrylate, poly(ethylene-vinylacetate), cellulose nitrite, silicones, poly (lactide-co-glycolide).

There are various ways that an excipient can be configured to control a dissolution profile of a sustained-release formulation. For example, the excipient can be intimately mixed with the drug (e.g., flecainide) in an amount effective for controlling release of the drug from the pharmaceutical formulation. Such a mixture can be in various forms, e.g., a dry mixture, a wet mixture, tablet, capsule, beads, etc., and may be formed in various ways. The resulting mixture can then be formed into the desired dosage form, e.g., tablet or capsule.

Effective amounts of controlled-release excipient(s) for controlling release may be determined by the guidance provided herein. For example, in some embodiments the sustained-release pharmaceutical formulation comprises at least about 5, 10, 15, 20, 25, 30, 35, 40, 45, 50, 55, 60, 65, 70, 75, 80, 85, 90, 95% (w/w) of the controlled-release excipient(s). In some embodiments, the concentration of the controlled-release excipient(s) in the pharmaceutical formulation may range about 5-95, 10-80, 20-70, 25-65, 35-55, 40-50, 5-20, 10-30, 20-40, 30-50, 40-60, 50-70, 60-80, 70-95% (w/w).

Various dissolution characteristics of the dissolution profile of the sustained-released flecainide pharmaceutical formulation can be controlled by appropriate configuration of the controlled-release excipient incorporated therein. Preferably, the dissolution profile comprises a dissolution rate that is slower than a dissolution rate of a comparable immediate-release flecainide formulation. For example, in some embodiments, the pharmaceutical formulation comprises flecainide and at least one controlled-release excipient configured to control an in vitro release profile within the following ranges of drug release: 0-40% released in 1 hour; 10-60% released in 4 hours; 20-80% released in 8 hours; >=70% released in 12 hours.

In an exemplary embodiment, the sustained-release pharmaceutical formulation comprises flecainide and at least one controlled-release excipient configured to provide, upon administration to a patient, an average free serum flecainide Cₘₐₓ value that is less than (e.g., at least about 5% less than) the average free serum flecainide Cₘₐₓ value of a comparable immediate-release flecainide under comparable conditions. For example, the controlled-release excipient can be configured to control an in vivo free flecainide serum profile wherein there is greater flecainide bioavailability, as indicated by an area under the serum concentration curve at steady state that is substantially equal to or greater than a conventional immediate-release flecainide formulation at the same dose, and a lower Cₘₐₓ at steady state than a conventional immediate-release flecainide formulation at the same dose.

Sustained-release flecainide pharmaceutical formulation as described herein may be formulated to be useful for oral administration under dosage schedules in the range of once or twice daily to once every two to seven days, to a subject having a condition or disorder for which the administration of flecainide is indicated. Thus, in some embodiments a pharmaceutical formulation comprises a controlled dosage form suitable for daily or weekly administration of flecainide.

Certain sustained-release flecainide formulations may exhibit one or more surprising and unexpected features and benefits. For example, sustained-release dosage forms are typically sought to enable longer time intervals between dosing of a drug having a short half-life in plasma, due for example to rapid metabolism, excretion or other routes of depletion.

In an embodiment, a method of treatment comprises administering a sustained-release pharmaceutical formulation as described herein to a patient in need thereof.

In some embodiments, the sustained-release pharmaceutical formulation is formed into capsules, tablets or other solid dosage forms suitable for oral administration. In embodiments, the sustained-release pharmaceutical formulation is formulated as a discrete solid dosage unit such as a tablet or capsule, wherein the flecainide or salt thereof is present therein as particles and is formulated together with one or more pharmaceutically acceptable excipients. In some embodiments the excipients are controlled-release excipients selected at least in part to provide a release profile and/or PK profile consistent with the desired profiles described herein.

In some embodiments, the particular solid dosage form selected is not critical so long as it achieves a release and/or PK profile as defined herein for the particular sustained-release formulation. In some embodiments the profile is achieved using one or more controlled-release excipients or release modifiers. In some embodiments release modifiers suitable for use include a wax or polymer matrix with which and/or in which the flecainide is dispersed; a release-controlling layer or coating surrounding the whole dosage unit or flecainide-containing particles, granules, beads or zones within the dosage unit.

Sustained-release pharmaceutical formulations can be configured in a variety of dosage forms, such as tablets and beads; can contain a variety of fillers and excipients, such as controlled-release excipients (also referred to a release modifiers); and may be made in a variety of ways. Those skilled in the art may determine the appropriate configuration by routine experimentation guided by the descriptions provided herein.

Sustained-release pharmaceutical formulations may contain fillers. Examples of suitable fillers include, but are not limited to, METHOCEL, methylcellulose, hydroxypropyl methylcellulose (HPMC), hydroxypropylcellulose (HPC), corn starch, polyvinyl alcohol (PVA), polyvinylpyrrolidone (PVP), cross-linked PVP, and the like.

Sustained-release flecainide pharmaceutical formulations may contain other excipients. Examples of suitable excipients include, but are not limited to, acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypryopl cellulose (L-HPC), magnesium stearate, maltodextrin, maltose, mannitol, methylcellulose, microcrystalline cellulose, methacrylate, sodium carboxymethylcellulose, polyvinyl acetate phthalate (PVAP), povidone, shellac, sodium starch glycolate, sorbitol, starch, sucrose, triacetin, triethylcitrate, vegetable based fatty acid, xanthan gum, xylitol, and the like.

In some embodiments, the sustained-release pharmaceutical formulation comprises, for example, from about 5%, 10%, 20% 30%, 40%, or 50%, to about 60%, 70%, 80%, 90% or 95% flecainide by weight. For example, in some embodiments the sustained-release pharmaceutical formulation comprises at least about 5, 10, 15, 20, 25, 30, 35, 40, 45, 50, 55, 60, 65, 70, 75, 80, 85, 90, 95% (w/w) of flecainide. In some embodiments, the concentration of flecainide in the pharmaceutical formulation may range from about 5-95, 10-80, 20-70, 25-65, 35-55, 40-50, 5-20, 10-30, 20-40, 30-50, 40-60, 50-70, 60-80, 70-95% (w/w).

The dissolution rate of the sustained-release flecainide pharmaceutical formulation determines how quickly flecainide becomes available for absorption into the blood stream and therefore controls the bioavailability of flecainide. Dissolution rate is dependent on the size and the composition of the dosage form. In some embodiments, the dissolution rate of the flecainide formulation can be changed by altering the additional components of the formulation. Disintegrants, such as starch or corn starch, or crosslinked PVPs, can be used to increase solubility when desired. Solubilizers can also be used to increase the solubility of the flecainide formulations. In some embodiments alternative binders, such as hydroxypropylmethyl cellulose (HPMC), hydroxypropyl cellulose (HPC), methyl cellulose (MC), PVP, gums, xanthine, and the like, can be used to increase the dissolution rate.

In some embodiments the dissolution rate of the formulation can be decreased by adding components that make the formulation more hydrophobic. For example, addition of polymers such as ethylcelluloses, wax, magnesium stearate, and the like can decrease the dissolution rate.

In some embodiments, the dissolution rate of the sustained-release pharmaceutical formulation is such that about 25% of the flecainide in the dosage form is dissolved within the first hour, about 60% of the flecainide is dissolved within the first 6 hours, about 80% of the flecainide is dissolved within the first 9 hours, and substantially all of the flecainide is dissolved within the first 12 hours. In other embodiments, the dissolution rate of the sustained-release pharmaceutical formulation is such that about 35% of the flecainide in the dosage form is dissolved within the first hour, about 85% of the flecainide is dissolved within the first 6 hours, and substantially all of the flecainide is dissolved within the first 9 hours. In yet other embodiments, the dissolution rate of the sustained-release pharmaceutical formulation in the dosage form is such that about 45% of the flecainide is dissolved within the first hour, and substantially all of the flecainide is dissolved within the first 6 hours.

The dissolution rate of the formulation can also be slowed by coating the dosage form. Examples of coatings include enteric coatings, sustained-release polymers, and the like.

The sustained-release pharmaceutical formulation can take about, for example, from 2, 4, 6, or 8 hours to about 15, 20, or 25 hours to dissolve. Preferably, the formulation has a dissolution rate of from about 3, 4, 5, or 6 to about 8, 9, or 10 hours.

Another embodiment provides a method of preparing (i e manufacturing) sustained-release pharmaceutical formulations. The method comprises mixing flecainide with an excipient and/or filler to form a mixture, and forming a suitable dosage form (e.g., tablet, capsule, bead, etc.) from the mixture. In some embodiments, the method of preparing the formulation further comprises adding another excipient and/or filler to the mixture prior to forming the dosage form. The filler and excipient are as described herein. In an embodiment, the flecainide is mixed with the filler and/or excipient to form a wet mixture. The wet mixture can then be formed into particles or beads, which can then be dried. The dried product can then be tableted or placed into a gelatin capsule for oral delivery.

In an embodiment, a pharmaceutical formulation comprises a sustained-release flecainide and a filler. In some embodiments the formulation further comprises an excipient. In some embodiments the filler is a polymer. In some embodiments the excipient is a polymer. In some embodiments the filler is selected from the group consisting of methylcellulose, hydroxypropyl methylcellulose (HPMC), hydroxypropylcellulose (HPC), corn starch, polyvinyl alcohol (PVA), polyvinylpyrrolidone (PVP), and cross-linked PVP. In some embodiments the excipient is selected from the group consisting of acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypropyl cellulose (L-HPC), magnesium stearate, maltodextrin, maltose, mannitol, methylcellulose, microcrystalline cellulose, methacrylate, sodium carboxymethylcellulose, polyvinyl acetate phathalate (PVAP), povidone, shellac, sodium starch glycolate, sorbitol, starch, sucrose, triacetin, triethylcitrate, vegetable based fatty acid, xanthan gum, and xylitol.

The invention also provides a pharmaceutical composition comprising compounds of the invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Pharmaceutically acceptable carriers include solvents, dispersion media, buffers, coatings, antibacterial and antifungal agents, wetting agents, preservatives, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

During the manufacturing, the carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Effective doses of the compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

In another aspect, more than one rate control agent may be administered, either incorporated into the same composition or administered as separate compositions. This can include any combination of rate control agents as individually described herein.

The flecainide described herein may be administered alone, or in combination with one or more rate control agent. In combinations, the rate control agent may be conjugated to the flecainide, incorporated into the same composition as the flecainide, or may be administered as a separate composition. The rate control agent may be administered prior to, during and/or after the administration of the flecainide.

In one embodiment, the flecainide is co-administered with the rate control agent. In another embodiment, the flecainide is administered independently from the administration of the rate control agent. In one embodiment, the flecainide is administered first, followed by the administration of the rate control agent. In another embodiment, the rate control agent is administered first, followed by the administration of flecainide.

The administration of the flecainide with other agents (e.g., a beta blocker drug, calcium channel blocker, digitalis, etc.) and/or treatments may occur simultaneously, or separately, via the same or different route, at the same or different times. Dosage regimens may be adjusted to provide the optimum desired response (e.g., a therapeutic or prophylactic response).

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

In one aspect, the dosage of flecainide may range from about 1 mg to about 4 g. In a particular embodiment, the dosage of flecainide may range from about 3 mg to about 1000 mg. In some suitable embodiments the drug is given in divided doses. In some suitable embodiments of the invention, 50-500 mg of the flecainide is administered. In one example, 50, 100, 150, 200, 300, 400, or 500 mg of the flecainide can be administered.

In another aspect, the dosage of a rate control agent, such as a beta blocker or calcium channel blocker, may range from about 1 mg to about 4 g. In a particular embodiment, the dosage of a rate control agent may range from about 3 mg to about 1000 mg. In some suitable embodiments the drug is given in divided doses. In some suitable embodiments of the invention, 10-500 mg of a rate control agent is administered. In some suitable embodiments of the invention, 50, 100, 200, 300, 400, or 500 mg of a rate control agent is administered.

In another aspect, the dosage of a rate control agent that is a digitalis drug may range from about 10 μg to about 1000 μg. In a particular embodiment, the dosage of a digitalis drug may range from about 50 μg to about 300 μg. In some suitable embodiments the drug is given in divided doses. In some suitable embodiments of the invention, 100-250 μg of a digitalis drug is administered. In some suitable embodiments of the invention, 50, 100, 125, 200, 300, 400, or 500 μg of a digitalis drug is administered.

In another aspect, the dosage of another agent useful in the treatment of a disease may include a therapeutically effective or clinically acceptable amount. In another example, the dosage of another agent is an amount that complements with or enhances the effect of a flecainide described herein.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated, or the form of sustained release technology employed. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, oral administration (for example, in capsules or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

In embodiments, patient compliance with a flecainide treatment can be much improved by administration in a sustained-release formulation. In embodiments, a feature of a sustained-release flecainide formulation can be the more effective control of free fraction flecainide in serum.

In some embodiments, the sustained release pharmaceutical formulation includes an effective amount of one or more other therapeutic agents, for example, but not limited to, a beta blocker drug, a calcium channel blocker, and a digitalis. Examples of a beta blocker drug include, but not limited to, atenolol, propranolol, bisoprolol, metoprolol, nadolol, acebutolol, betaxolol, bisoprolol, celiprolol, esmolol, metoprolol, nebivolol, alprenolol, bucindolol, carteolol, carvedilol, labetalol, levobunolol, medroxalol, mepindolol, metipranolol, nadolol, oxprenolol, penbutolol, pindolol, propafenone (propafenone is a sodium channel blocking drug that also is a beta-adrenergic receptor antagonist), propranolol, sotalol, and timolol. Examples of a calcium channel blocker include, but not limited to, Dihydropyridines (e.g. amlodipine), benzothiapines (e.g. diltiazem), and phenylalkylamines (e.g. verapamil), felodipine, nifedipine.

The formulations described herein can be used to treat any suitable mammal, including primates, such as monkeys and humans, horses, cows, cats, dogs, rabbits, and rodents such as rats and mice. In one embodiment, the mammal to be treated is human.

The invention further provides kits that comprise a therapeutically effective amount of a flecainide and a therapeutically effective amount of a rate control agent described herein, along with instructions for administration. In some embodiments, the beta blocker drug is present in said kit in an amount effective to control the release rate of flecainide to treat a heart disease.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

### Combination of Flecainide and Beta Blocker

Exemplary formulations comprising flecainide and a beta blocker, consistent with the description provided above, are shown in Tables 1-4 and are prepared using the methods described herein.

The exemplified formulation is a therapeutic tablet for oral administration. The formulation includes a mixture of a flecainide and a beta blocker drug. The formulation also includes pharmaceutical grade excipients.

TABLE 1
Exemplary formulation having flecainide and propranolol.

| Ingredients | Concentration (% w/w) |
|---|---|
| Flecainide 150 | 50 |
| Propranolol 60 mg | 30 |
| Binders and inert ingredients | 20 |
| Total | 100 |

TABLE 2
Exemplary formulation having flecainide and metoprolol.

| Ingredients | Concentration (% w/w) |
|---|---|
| Flecainide 150 mg | 50 |
| Metoprolol 50 mg | 25 |
| Binders and inert ingredients | 25 |
| Total | 100 |

TABLE 3
Exemplary formulation having flecainide and atenolol.

| Ingredients | Concentration (% w/w) |
|---|---|
| Flecainide 150 mg | 50 |
| Atenolol 50 mg | 20 |
| Binders and inert ingredients | 30 |
| Total | 100 |

TABLE 4
Exemplary formulation having flecainide and bisoprolol.

| Ingredients | Concentration (% w/w) |
|---|---|
| Flecainide 150 | 50 |
| Bisoprolol 50 mg | 25 |
| Binders and inert ingredients | 25 |
| Total | 100 |

### Example 2

### Combination of Flecainide and Calcium Channel Blocker

Exemplary formulations comprising flecainide and a calcium channel blocker, consistent with the description provided above, are shown in Tables 5-6 and are prepared using the methods described herein.

The formulation is a therapeutic tablet for oral administration. The formulation includes a mixture of a flecainide and a calcium channel blocker. The formulation also includes pharmaceutical grade excipients.

TABLE 5
Exemplary formulation having flecainide and Verapamil.

| Ingredients | Concentration (% w/w) |
|---|---|
| Flecainide 300 mg | 50 |
| Verapamil 300 mg | 50 |
| Total | 100 |

TABLE 6
Exemplary formulation having flecainide and Diltiazem.

| Ingredients | Concentration (% w/w) |
|---|---|
| Flecainide 300 mg | 50 |
| Diltiazem 360 mg | 50 |
| Total | 100 |

### Example 3

### Combination of Flecainide and Digitalis

Exemplary formulations comprising flecainide and a digitalis, consistent with the description provided above, are shown in Tables 7-8 and are prepared using the methods described herein.

The formulation is a therapeutic tablet for oral administration. The formulation includes a mixture of a flecainide and a digitalis drug. The formulation also includes pharmaceutical grade excipients.

TABLE 7
Exemplary formulation having flecainide and propranolol.

| Ingredients | Concentration (% w/w) |
|---|---|
| Flecainide 150 mg | 50 |
| Digitoxin 0.2 mg | 25 |
| Binders and inert ingredients | 25 |
| Total | 100 |

TABLE 8
Exemplary formulation having flecainide and sotalol.

| Ingredients | Concentration (% w/w) |
|---|---|
| Flecainide 150 mg | 50 |
| Digoxin 0.125 mg | 25 |
| Binders and inert ingredients | 25 |
| Total | 100 |

### Examples 4-5

### Sustained Release Flecainide Formulations

Exemplary process that can be employed for preparation of a sustained release flecainide formulation consistent with the description provided above, and an exemplary formulation that can be prepared, are shown and described below.

### Example 4

The following formulation method is an example of preparation of a slow-release flecainide formulation. Wet granulation, extrusion, and fluid-bed drying processes can be utilized to produce sustained-release flecainide particles or pellets.

To prepare the wet granules, flecainide, microcrystalline cellulose (Avicel PH 102) and methylcellulose (Methocel A15 LV), at the various percentages, can be placed into a high-shear granulator and mixed for 15 minutes. Deionized (DI) water can be added slowly, and the wet granules can be mixed for another 5-10 minutes.

The pellets can then be dried using a fluid bed dryer. The dried pellets can be discharged from the fluid-bed dryer and be sized by passing through different screens.

The dried pellets can then be encapsulated into hard gelatin capsules.

### Example 5

A PLGA copolymer is provided. Flecainide can be loaded into the PLGA copolymer. The formulation may be in the form of tablet or capsule.

The formulations described in Example 4 and 5 can be orally administered to a subject.

Serum can be collected and analyzed. The flecainide composition may achieve a therapeutic effect within 2 hrs and maintain therapeutic effect for at least 24 hours in >95% percent of treated patients.

The composition may allow for consistent release of the active agent from the drug delivery vehicle with no more than 25% variation plus an encapsulation efficiency of over 70%. The composition may release the active agent from the drug delivery vehicle with >85% intact over the entire duration of release.

Having described preferred embodiments of the invention, it is to be understood that the invention is not limited to the precise embodiments, and that various changes and modifications may be effected therein by those skilled in the art without departing from the scope or spirit of the invention as defined in the appended claims.

1. A pharmaceutical formulation comprising a flecainide in combination with a rate control agent, wherein the rate control agent comprises a β blocker, and wherein the β blocker is metoprolol.

2.-11. (canceled)

12. The pharmaceutical formulation of claim 1, wherein the flecainide comprises a flecainide acetate.

13. The pharmaceutical formulation of claim 1, configured in a dosage form selected from once daily, twice daily, once every two days, once every three days, once every four days, once every five days, once every six days, and once weekly.

14. The pharmaceutical formulation of claim 1, wherein the flecainide is present in an amount of about 50 to about 500 mg.

15. The pharmaceutical formulation of claim 1, wherein the rate control agent is present in an amount of about 50 to about 500 mg.

16. The pharmaceutical formulation of claim 1, wherein the formulation further comprises an effective amount of one or more additional therapeutic agents.

17. A method of treating atrial fibrillation, atrial flutter, and/or supraventricular tachycardia (SVT) in a subject, the method comprising administering the pharmaceutical formulation of claim 1 to a subject in need thereof.

18.-35. (canceled)

36. A controlled-release pharmaceutical formulation comprising a flecainide and a controlled-release excipient.

37. The controlled-release pharmaceutical formulation of claim 36 wherein the controlled-release formulation comprises a once daily sustained-release formulation.

38. The controlled-release pharmaceutical formulation of claim 36, wherein the controlled-release excipient is configured to modify a dissolution profile of said sustained-release flecainide.

39. The controlled-release pharmaceutical formulation of claim 38, wherein the controlled-release excipient comprises at least one selected from hydroxypropyl methylcellulose (HPMC), hydroxyethylcellulose, hydroxypropylcellulose (HPC), methylcellulose, ethylcellulose, cellulose acetate butyrate, cellulose acetate phthalate, hydroxypropylmethyl cellulose phthalate, microcrystalline cellulose, corn starch, polyethylene oxide, polyvinyl alcohol (PVA), polyvinylpyrrolidone (PVP), cross-linked PVP, polyvinyl acetate phthalate, polyethylene glycol, zein, poly-DL-lactide-co-glycolide (PLGA), dicalcium phosphate, calcium sulfate, and mixtures thereof.

40. The controlled-release pharmaceutical formulation of claim 36, wherein said flecainide is a flecainide acetate.

41. The controlled-release pharmaceutical formulation of claim 36, further comprising a filler wherein said filler comprises at least one selected from acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypryopl cellulose (L-HPC), magnesium stearate, maltodextrin, maltose, mannitol, methylcellulose, microcrystalline cellulose, methacrylate, sodium carboxymethylcellulose, polyvinyl acetate phthalate (PVAP), povidone, shellac, sodium starch glycolate, sorbitol, starch, sucrose, triacetin, triethylcitrate, vegetable based fatty acid, xanthan gum, and xylitol.

42. The controlled-release pharmaceutical formulation of claim 36, configured in a dosage form selected from once daily, twice daily, once every two days, once every three days, once every four days, once every five days, once every six days, and once weekly.

43. The controlled-release pharmaceutical formulation of claim 36, wherein the flecainide is present in an amount of about 50 to about 500 mg.

44. The controlled-release pharmaceutical formulation of claim 36, wherein the formulation further comprises an effective amount of one or more other therapeutic agents.

45. The controlled-release pharmaceutical formulation of claim 44, wherein at least one of said other therapeutic agent comprises a rate control agent.

46. The controlled-release pharmaceutical formulation of claim 45, wherein said rate control agent comprises a beta blocker.

47. The controlled-release pharmaceutical formulation of claim 45, wherein said rate control agent comprises a calcium channel blocker.

48. The controlled-release pharmaceutical formulation of claim 45, wherein said rate control agent comprises a digitalis.

49. A method of treating atrial fibrillation, atrial flutter, and/or supraventricular tachycardia (SVT) in a subject, comprising administering the pharmaceutical formulation of claim 36 to a subject in need thereof.

50.-53. (canceled)

54. The controlled-release pharmaceutical formulation of claim 36, wherein the flecainide is dispersed in a wax matrix.

55. The controlled-release pharmaceutical formulation of claim 36, wherein the flecainide is dispersed in a polymer matrix.

56. The controlled-release pharmaceutical formulation of claim 36, wherein the flecainide is dispersed in an encapsulated form.
